(19) United States
(12) Patent Application Publication
(10) Pub. No.: US 2009/0035340 A1
(43) Pub. Date: Feb. 5, 2009

(54) PRESERVATIVE COMPOSITIONS FOR MOIST WIPES

(75) Inventors: Andrew Sjaak Landa, Appleton, WI (US); Jose Huerta, Appleton, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: KIMBERLY-CLARK WORLDWIDE, INC., Neenah, WI (US)

(21) Appl. No.: 11/830,204

(22) Filed: Jul. 30, 2007

(51) Int. Cl. A61K 31/426; A61K 9/70

(52) U.S. Cl. 424/405; 514/372

(57) ABSTRACT

The present disclosure generally relates to a composition for use with personal care products. The composition comprises a preservative system comprising methylisothiazolinone and a benzoic acid salt or derivative thereof. The weight ratio of methylisothiazolinone and benzoic acid salt in the composition is from about 1:30 to about 1:100. Also disclosed are personal care products, such as wet wipes, impregnated with the composition.

### BACKGROUND OF DISCLOSURE

The present disclosure generally relates to a composition for use with personal care products. The composition comprises a preservative system comprising methylisothiazolinone and a benzoic acid salt or derivative thereof. The weight ratio of methylisothiazolinone and benzoic acid salt in the composition is from about 1:30 to about 1:100. Also disclosed are personal care products, such as wet wipes, impregnated with the composition.

Personal care products come in many different forms and may include, for example, creams, lotions, pastes, liquids, shampoos, absorbent products, and wet wipes, among others. For purposes of product safety, it is generally necessary that the product contain a preservative which is effective for inactivating any microorganisms which may be introduced into the product prior to its use during manufacturing or storage. Furthermore, since personal care products may be used to directly contact skin or mucosa such as around body orifices where the potential for transfer of materials from the product to the consumer may be a concern, it is generally good practice to reduce contamination of the product in every possible way. The need to control microbiological growth in personal care products is particularly acute in water based products such as non-ionic oil-in-water emulsions and in pre-impregnated wipes such as wet wipes.

In addition to avoiding health concerns, preventing the growth and proliferation of microorganisms in the product will also increase the useful shelf life of the product, and prevent off-color, off-odor, or destruction of the product components resulting from contamination by microorganisms.

There are numerous available preservatives that may be incorporated into personal care products to help keep microbiological and fungal growth at an acceptable level. In some cases, however, commercial preservatives cannot provide effective control over a broad spectrum of microorganisms, even at high use concentrations, due to weak activity against certain types of microorganisms. Other preservatives may not be suitable for use in combination with products designed to contact human skin or mucosa, such as personal care products. Furthermore, federal guidelines may limit the amounts of certain preservatives that can be included in personal care products. There are thus limits to the amounts and types of preservatives that may be used to control contamination by microorganisms in personal care products.

The present disclosure addresses these problems by providing a preservative system that may be used in combination with personal care products. The preservative system comprises a combination of preservatives that together have efficacy against a broad spectrum of microorganisms.

### SUMMARY OF THE DISCLOSURE

The present disclosure generally relates to a composition for use with personal care products. The composition comprises a preservative system comprising methylisothiazolinone and a benzoic acid salt or derivative thereof. The weight ratio of methylisothiazolinone and benzoic acid salt in the composition is from about 1:30 to about 1:100. Also disclosed are personal care products, such as wet wipes, impregnated with the composition.

In one aspect, the present disclosure is directed to a composition comprising methylisothiazolinone and a benzoic acid salt or derivative thereof at a weight ratio of methylisothiazolinone to benzoic acid salt or derivative thereof of from about 1:30 to about 1:100, wherein the composition has a pH of about 6.0 or less.

In another aspect, the present disclosure is directed to a wet wipe comprising a wipe substrate and a liquid composition, the liquid composition comprising methylisothiazolinone and a benzoic acid salt or derivative thereof at a weight ratio of methylisothiazolinone to benzoic acid salt of from about 1:30 to about 1:100, wherein the liquid composition has a pH of about 6.0 or less.

Other objects and features will be in part apparent and in part pointed out hereinafter.

### DETAILED DESCRIPTION OF THE DISCLOSURE

The present disclosure generally relates to a composition for use with personal care products. The composition comprises a preservative system comprising methylisothiazolinone and a benzoic acid salt or derivative thereof. The weight ratio of methylisothiazolinone and benzoic acid salt in the composition is from about 1:30 to about 1:100. Also disclosed are personal care products, such as wet wipes, impregnated with the composition.

In accordance with the present disclosure, it has been discovered that methylisothiazolinone in combination with a benzoic acid salt or derivative thereof provides enhanced microbicidal efficacy over current preservative packages. In particular, the combination of methylisothiazolinone and a benzoic acid salt or derivative thereof has been found to be an effective microbicide for a broad spectrum of bacteria and fungi, including yeast and molds. As used herein, the term “microbicide” refers to a compound capable of killing, inhibiting the growth of, or controlling the growth of microorganisms. Microbicides include bactericides, fungicides, and algaecides. The term “microorganism” includes, for example, fungi (including yeast and mold), bacteria, and algae.

Thus, in one aspect, the present disclosure is directed to a preservative system comprising methylisothiazolinone and a benzoic acid salt or derivative thereof, such as sodium benzoate. The combination of methylisothiazolinone and benzoic acid salt or derivative thereof provides enhanced preservative efficacy as compared to either preservative alone. Although both methylisothiazolinone and benzoic acid salts or derivatives are broad spectrum antimicrobials, these two preservatives exert their microbicidal effects by distinct mechanisms, allowing for a greater overall antimicrobial effect when used in combination. For instance, methylisothiazolinone exhibits very good antibacterial effects, while the benzoic acid salts and derivatives described herein are particularly effective against molds and yeast.

Additionally, the preservative system is advantageously non-harmful to skin and mucosa in the concentrations set forth herein, and may thus be formulated with a suitable pharmaceutically acceptable carrier into compositions such as lotions, creams, liquids, and the like, that may be applied to skin or mucosa. The presence of the preservatives in the compositions provides the compositions with improved microbicidal properties against most bacteria and fungi, as well as improving the composition shelf life. The preservative-containing compositions of the present disclosure advantageously meet the CTFA guidelines for preservative efficacy for wet wipes, and in the case of some microorganisms, exceed these guidelines, as can be seen from the examples set forth herein.

In another aspect, a preservative-containing composition of the present disclosure may be used in combination with a product, such as a personal care product. More particularly, the composition may be incorporated into or onto a substrate, such as a wipe substrate, an absorbent substrate, a fabric or cloth substrate, or a tissue substrate, among others. For example, the preservative-containing compositions may be incorporated into personal care products, such as wipes, absorbent articles, bath tissues, cloths, and the like. In one preferred embodiment, the preservative-containing composition is a liquid composition that may be used in combination with a wipe substrate to form a wet wipe.

Typically, the methylisothiazolinone and benzoic acid salt or derivative thereof are present in the compositions in a weight ratio of methylisothiazolinone to benzoic acid salt or derivative thereof of from about 1:30 to about 1:100, preferably from about 1:40 to about 1:85, and more preferably about 1:50 to about 1:70.

Preferably, the amount of methylisothiazolinone in the preservative-containing composition is from about 0.005% (w/w) to about 0.01% (w/w), more preferably is present in the composition in an amount of from about 0.0070% (w/w) to about 0.0095% (w/w). It should be noted that when the preservative-containing composition is incorporated into a personal care product, for example, is a liquid (or wetting) composition for use in a wet wipe, the amount of methylisothiazolinone is by total weight of the composition.

The amount of benzoic acid salt or derivative thereof in the preservative-containing composition is preferably from about 0.30% (w/w) to about 0.50% (w/w), more preferably is from about 0.35% (w/w) to about 0.50% (w/w), and still more preferably is from about 0.40% (w/w) to about 0.50% (w/w). It should be noted that when the preservative-containing composition is incorporated into a personal care product, for example, is a liquid (or wetting) composition for use in a wet wipe, the amount of benzoic acid salt or derivative thereof is by total weight of the composition.

In one preferred embodiment, the composition comprises from about 0.005% (w/w) to about 0.01% (w/w) methylisothiazolinone and from about 0.30% (w/w) to about 0.50% (w/w) benzoic acid salt or derivative thereof.

The benzoic acid salt or derivative thereof used herein may be any salt or derivative of benzoic acid that has microbicidal properties, and preferably is selected from the group consisting of sodium benzoate, potassium benzoate, and combinations thereof. Preferably, the benzoic acid salt or derivative thereof is soluble in water at a pH of about 6.0 or less.

As noted above, the preservative system of the present disclosure may be formulated with one or more conventional pharmaceutically-acceptable and compatible carrier materials to form a preservative-containing composition. The preservative-containing composition may take a variety of forms including, without limitation, aqueous solutions, gels, balms, lotions, suspensions, creams, milks, salves, ointments, sprays, emulsions, oils, resins, foams, solid sticks, aerosols, and the like. Carrier materials suitable for use in the instant disclosure include those well-known for use in the cosmetic and medical arts as a basis for ointments, lotions, creams, salves, aerosols, gels, suspensions, sprays, foams, and the like, and may be used in their art-established levels.

Non-limiting examples of suitable carrier materials include water, emollients, sterols or sterol derivatives, natural and synthetic fats or oils, viscosity enhancers, rheology enhancers, polyols, surfactants, alcohols, esters, silicones, clays, starch, cellulose, and other pharmaceutically acceptable carrier materials. As will be recognized by one skilled in the art, the relative amounts of components in the compositions of the disclosure that can be used to formulate the composition will be dictated by the nature of the composition. The levels can be determined by routine experimentation in view of the disclosure provided herein.

Thus, in one embodiment, the preservative-containing composition of the disclosure can optionally include one or more emollient, which typically acts to soften, soothe, and otherwise lubricate and/or moisturize the skin. Suitable emollients that can be incorporated into the compositions include oils such as petrolatum based oils, petrolatum, vegetable based oils, mineral oils, natural or synthetic oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof.

The esters can be selected from cetyl palmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof can also suitably be used as emollients.

The composition may desirably include one or more emollient in an amount of from about 0.01% (w/w) to about 20% (w/w), more desirably from about 0.05% (w/w) to about 5% (w/w), and even more desirably from about 0.10% (w/w) to about 2% (w/w).

Stearol and stearol derivatives which are suitable for use in the compositions of the present disclosure include, but are not limited to cholestol, sitosterol, stigmasterol, ergosterol, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyidecanoate, ergocalciferol, tall oil sterol, soy sterol acetate, lanasterol, soy sterol, avocado sterols, fatty alcohols, and combinations thereof.

The composition of the invention can desirably include sterols, sterol derivatives or mixtures of both sterols and sterol derivatives in an amount of from about 0.01% (w/w) to about 10% (w/w), more desirably from about 0.05% (w/w) to about 5% (w/w), and even more desirably from about 0.1% (w/w) to about 1% (w/w).

The compositions of the disclosure can also include natural fats and oils. As used herein, the term “natural fat or oil” is intended to include fats, oils, essential oils, essential fatty acids, non-essential fatty acids, phospholipids, and combinations thereof. These natural fats and oils can provide a source of essential and non-essential fatty acids to those found in the skin's natural barrier. Suitable natural fats or oils can include citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, soybean oil, and combinations thereof.

The composition of the invention may desirably include fats and oils in an amount of from about 0.01% (w/w) to about 20% (w/w), more desirably from about 0.05% (w/w) to about 10% (w/w), and even more desirably from about 0.1% (w/w) to about 5% (w/w).

Optionally, one or more viscosity enhancers may be added to the composition to increase the viscosity, to help stabilize the composition, such as when the composition is incorporated into a personal care product, thereby reducing migration of the composition and improve transfer to the skin. Suitable viscosity enhancers include polyolefin resins, lipophilic/oil thickeners, ethylene/vinyl acetate copolymers, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, clays, carbomers, acrylic based thickeners, and combinations thereof.

The composition may desirably include one or more viscosity enhancers in an amount of from about 0.01% (w/w) to about 25% (w/w), more desirably from about 0.05% (w/w) to about 10% (w/w), and even more desirably from about 0.1% (w/w) to about 5% (w/w).

The compositions of the disclosure may optionally further comprise rheology enhancers. Rheology enhancers may help increase the melt point viscosity of the composition so that the composition readily remains on the surface of a personal care product and does not substantially migrate into the interior of the product, while substantially not affecting the transfer of the composition to the skin. Additionally, the rheology enhancers help the composition to maintain a high viscosity at elevated temperatures, such as those encountered during storage and transportation.

Suitable rheology enhancers include combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, and styrene-ethylene/propylene copolymers and combinations thereof. Specifically, rheology enhancers such as mineral oil and ethylene/propylene/styrene copolymers, and mineral oil and butylene/ethylene/styrene copolymers (Versagel blends from Penreco) are particularly preferred. Also, Vistanex (Exxon) and Presperse (Amoco) polymers are particularly suitable rheology enhancers.

The composition of the invention can suitably include one or more rheology enhancer in an amount of from about 0.1% (w/w) to about 5% (w/w).

In one preferred embodiment, the preservative-containing compositions may comprise water. For instance, where the preservative-containing composition is a wetting composition, such as described below for use with a wet wipe, the composition will typically comprise water. The compositions can suitably comprise water in an amount of from about 0.1% (w/w) to about 99% (w/w), more preferably from about 10% (w/w) to about 90% (w/w), and still more preferably from about 30% (w/w) to about 85% (w/w).

The compositions may also comprise organic or inorganic salts. In instances where the preservative-containing composition is a wetting composition, such as described below for use with a dispersible wet wipe, the salts present in the composition may be insolubilizing agents and help stabilize the wipe basesheet in the presence of the wetting composition. Examples of suitable salts include sodium chloride, magnesium sulfate, sodium sulfate, magnesium chloride, sodium methyl sulfate zinc chloride, ammonium sulfate, sodium aspartate, sodium succinate, citric acid trisodium salt, monosodium glutamate, sodium malate, sodium lactate, aspartic acid, sodium gluconate, calcium citrate malate, calcium lactate, magnesium aspartate, magnesium succinate, magnesium citrate (1:1), magnesium citrate (3:2), magnesium citrate malate, magnesium sodium citrate, magnesium malate, magnesium glutamate, magnesium lactate, magnesium gluconate, glycine, alanine, serine, betaine, taurine, poly(MAH) sodium salt, poly(ethylene-alt-maleic acid) magnesium salt, poly(0.45 MAH/0.55 AA) sodium salt, polyvinyl amine formate, poly(vinyl alcohol), poly(acrylic acid) sodium salt, poly(ethylene-alt-maleic acid) sodium salt, and combinations thereof. In these embodiments, the compositions can suitably comprise salts in amounts of from about 0.1% (w/w) to about 10.0% (w/w), and preferably about 0.5% (w/w) to about 5.0% (w/w).

In other embodiments, the compositions may optionally comprise acids, such as malic acid. Acids may be included in the composition to adjust the composition's pH to the desired level. The compositions can thus suitably comprise acids in amounts of from about 0.01% (w/w) to about 0.5% (w/w), more preferably from about 0.05% (w/w) to about 0.30% (w/w).

In order to maintain the maximum effectiveness of the preservatives, it is generally preferably for the preservative-containing compositions described herein to have a pH of about 6.0 or less. More preferably, the preservative-containing compositions have a pH of from about 4.2 to about 5.8, and more preferably about 4.5 to about 5.5. Although the preservative system will still be effective at pHs below about 4.2, in instances where the preservative-system is incorporated into products that contact skin, it is typically preferable for the pH of the preservative-containing compositions to be about 4.2 or greater to avoid damaging or irritating the skin.

It should be noted that the pH values set forth herein refer to the pH of the compositions that will come in contact with a user's body (e.g., skin). For instance, as noted elsewhere herein, the preservative system may be incorporated into a wetting composition for use with a wet wipe. However, other wipe components, such as basesheet components or additives, may somewhat alter the overall pH of the preservative-containing wetting composition. Thus, the pH of the preservative-containing compositions described herein may be somewhat different prior to contact with a wipe substrate or incorporation into other personal care products.

The compositions of the present disclosure may additionally include adjunct components conventionally found in pharmaceutical compositions in their art-established fashion and at their art-established levels. For example, the compositions may comprise additional compatible pharmaceutically active materials for combination therapy, such as antimicrobials, antioxidants, anti-parasitic agents, antipruritics, antifungals, antiseptic actives, biological actives, astringents, keratolytic actives, local anaesthetics, anti-stinging agents, anti-reddening agents, skin soothing agents, and combinations thereof. Other suitable additives that may be included in the compositions of the present disclosure include colorants, deodorants, fragrances, perfumes, emulsifiers, anti-foaming agents, lubricants, natural moisturizing agents, skin conditioning agents, skin protectants and skin benefit agents (e.g., aloe vera and laponite), solvents, solubilizing agents, suspending agents, wetting agents, humectants, preservatives, propellants, dyes and/or pigments, and combinations thereof.

In another aspect, the preservative-containing compositions of the present disclosure may be used in combination with a product, such as a personal care product. More particularly, the composition may be incorporated into or onto a substrate, such as a wipe substrate, an absorbent substrate, a fabric or cloth substrate, or a tissue substrate, among others. For example, the compositions may be incorporated into personal care products, such as wipes, absorbent articles, bath tissues, cloths, and the like. More particularly, the preservative-containing composition may be incorporated into wipes such as wet wipes, hand wipes, face wipes, cosmetic wipes, and the like, or absorbent articles, such as diapers, training pants, adult incontinence products, feminine hygiene products, and the like. In one preferred embodiment, the preservative-containing composition is a liquid composition that may be used in combination with a wipe substrate to form a wet wipe or may be a wetting composition for use in combination with a dispersible wet wipe.

Although discussed primarily in terms of a wetting composition for use in a dispersible wet wipe, it should be understood that the preservative-containing compositions described herein can also be used in combination with other non-dispersible wet wipes and numerous personal care products, such as those described above.

Thus, in one particularly preferred embodiment, the preservative system is incorporated into a wetting composition for use in a dispersible wet wipe. The dispersible wet wipe may desirably be adhesively bonded with a triggerable polymer. The term “triggerable” refers to the ability of this polymer to selectively provide the wet wipe with the desired in-use strength, while also providing it with the ability to lose sufficient strength such that the wet wipe will disperse when disposed in tap water, such as is found in toilets for example.

The wet wipe may comprise a nonwoven material that is wetted with an aqueous solution termed the “wetting composition,” which may also comprise the preservative system disclosed herein. The nonwoven material may comprise either a nonwoven fabric or a nonwoven web. The nonwoven fabric may comprise a fibrous material, while the nonwoven web may comprise the fibrous material and a binder composition.

The wetting composition desirably maintains the insolubility of the binder composition and may comprise an aqueous composition containing an insolubilizing agent. When the wet wipe is exposed to tap water, the wetting composition dilutes and the binder composition desirably loses strength leading to concomitant fragmentation and dispersal of the wet wipe. Thus, the combination of the binder composition and the wetting composition preferably affords the structural integrity or coherency necessary to maintain the in-use strength and properties of the wet wipe, while also allowing for selective fragmentation or dispersal of the wet wipe under desired conditions.

The nonwoven web of the wet wipe may be generated by spraying the fibrous material with the binder composition, wherein the binder composition comprises a mixture or solution comprising the triggerable polymer and optionally other components such as cobinders and/or an anti-blocking agent.

### Nonwoven Material

In many personal care products, nonwoven materials are the preferred substrate, especially with regard to wet wipes. As previously discussed, two types of nonwoven materials are described herein, the “nonwoven fabrics” and the “nonwoven webs”. As used herein, the nonwoven fabric comprises a fibrous material or substrate, where the fibrous material or substrate comprises a sheet that has a structure of individual fibers or filaments randomly arranged in a mat-like fashion and does not include the binder composition. Nonwoven fabrics may be made from a variety of processes including, but not limited to, airlaid processes, wet-laid processes such as with cellulosic-based tissues or towels, hydroentangling processes, staple fiber carding and bonding, and solution spinning.

Since nonwoven fabrics do not include a binder composition, the fibrous substrate used for forming the nonwoven fabric may desirably have a greater degree of cohesiveness and/or tensile strength than the fibrous substrate that is used for forming the nonwoven web. For this reason nonwoven fabrics comprising fibrous substrates created via hydroentangling may be particularly preferred for formation of the nonwoven fabric. Hydroentangled fibrous materials may provide the desired in-use strength properties for wet wipes that comprise a nonwoven fabric.

With regard to the nonwoven web, the binder composition may be applied to the fibrous material or substrate to form the nonwoven web using a variety of techniques. The fibrous material used to form the nonwoven web, may desirably have a relatively low wet cohesive strength prior to its treatment with the binder composition. Thus, when the fibrous substrate is bonded together by the binder composition, the nonwoven web will preferably break apart when it is placed in tap water, such as found in toilets and sinks. Thus the identity of the fibrous material may depend on whether it is to be used to form the nonwoven fabric or the nonwoven web. Furthermore, the fibers from which the fibrous material is made may also be selected based on whether they are to be used for a nonwoven web or nonwoven fabric.

The fibers forming the fibrous material may be made from a variety of materials including natural fibers, synthetic fibers, and combinations thereof. The choice of fibers may depend upon, for example, the intended end use of the finished substrate, the fiber cost and whether fibers will be used for a nowoven fabric or a nonwoven web. For instance, suitable fibers may include, but are not limited to, natural fibers such as cotton, linen, jute, hemp, wool, wood pulp, etc. Similarly, suitable fibers may also include: regenerated cellulosic fibers, such as viscose rayon and cuprammonium rayon; modified cellulosic fibers, such as cellulose acetate; or synthetic fibers, such as those derived from polypropylenes, polyethylenes, polyolefins, polyesters, polyamides, polyacrylics, etc. Regenerated cellulose fibers, as briefly discussed above, include rayon in all its varieties as well as other fibers derived from viscose or chemically modified cellulose, including regenerated cellulose and solvent-spun cellulose, such as Lyocell. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Chemically treated natural cellulosic fibers may be used, such as mercerized pulps, chemically stiffened or crosslinked fibers, or sulfonated fibers.

In addition, cellulose produced by microbes and other cellulosic derivatives may be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. Blends of one or more of any of the previously described fibers may also be used, if so desired.

The fibrous material may be formed from a single layer or multiple layers. In the case of multiple layers, the layers are generally positioned in a juxtaposed or surface-to-surface relationship and all or a portion of the layers may be bound to adjacent layers. The fibrous material may also be formed from a plurality of separate fibrous materials wherein each of the separate fibrous materials may be formed from a different type of fiber. In those instances where the fibrous material includes multiple layers, the binder composition may be applied to the entire thickness of the fibrous material, or each individual layer may be separately treated and then combined with other layers in a juxtaposed relationship to form the finished fibrous material.

Airlaid nonwoven fabrics are particularly well suited for use as wet wipes. The basis weights for airlaid nonwoven fabrics may range from about 20 to about 200 grams per square meter (gsm) with staple fibers having a denier of about 0.5-10 and a length of about 6-15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

Processes for producing airlaid non-woven basesheets are described in, for example, published U.S. Pat. App. No. 2006/0008621, herein incorporated by reference.

### Binder Composition

In one embodiment the binder composition may comprise a triggerable polymer. In another embodiment, the binder composition may comprise the triggerable polymer, a cobinder polymer and/or an antiblocking agent, such as disclosed in Published U.S. Patent App. No. 2007/0141936, herein incorporated by reference in its entirety. In addition to providing the wet wipe with in-use strength in the presence of the wetting composition and selective dispersibility in tap water, the binder composition preferably possesses a variety of other desirable properties. For example, the binder composition may preferably be processable on a commercial scale (i.e., the binder may be capable of rapid application on a large scale, such as by spraying) and may also desirably be inexpensive. The binder composition may also desirably provide acceptable levels of sheet wettability. In addition, all components of the wet wipe, including the binder composition, may preferably be non-toxic and relatively economical.

The amount of binder composition present in the nonwoven web may desirably range from about 5 to about 65 percent by weight based on the total weight of the nonwoven web. More desirably, the binder composition may comprise from about 7 to about 35 percent by weight based on the total weight of the nonwoven web. Even more desirably, the binder composition may comprise from about 10 to about 25 percent by weight based on the total weight of the nonwoven web. Most desirably, the binder composition may comprise from about 15 to 20 percent by weight based on the total weight of the nonwoven web. The amount of the binder composition desirably results in a nonwoven web that has in-use integrity, but quickly disperses when soaked in tap water.

The composition of tap water can vary greatly depending on the water source. Thus the binder composition may preferably be capable of losing sufficient strength to allow the wet wipe to disperse in tap water covering the preponderance of the tap water composition range found throughout the United States (and throughout the world). Thus, it is important to evaluate the dispersibility of the binder composition in aqueous solutions which contain the major components in tap water and in a representative concentration range encompassing the majority of the tap water sources in the United States. The predominant inorganic ions typically found in drinking water are sodium, calcium, magnesium, bicarbonate, sulfate and chloride. Based on a recent study conducted by the American Water Works Association (AWWA) in 1996, the predominance of the U.S. municipal water systems (both ground water and surface water sources) surveyed have a total dissolved solids of inorganic components of about 500 ppm or less. This level of 500 ppm total dissolved solids also represents the secondary drinking water standard set by the U.S. Environmental Protection Agency. The average water hardness, which represents the calcium and magnesium concentrations in the tap water source, at this total dissolved solids level was ca. 250 ppm (CaCO₃ equiv.), which also encompasses the water hardness for the predominance of the municipal water systems surveyed by the AWWA. As defined by the United States Geological Survey (USGS), a water hardness of 250 ppm equiv. CaCO₃ would be considered “very hard” water. Similarly, the average bicarbonate concentration at 500 ppm total dissolved solids reported in the study was ca. 112 ppm, which also encompasses the bicarbonate, or alkalinity, of the predominance of the municipal water systems surveyed. A past study by the USGS of the finished water supplies of 100 of the largest cities in the United States suggests that a sulfate level of about 100 ppm is sufficient to cover the majority of finished water supplies. Similarly, sodium and chloride levels of at least 50 ppm each should be sufficient to cover the majority of U.S. finished water supplies. Thus, binder compositions which are capable of losing strength in tap water compositions meeting these minimum requirements should also lose strength in tap water compositions of lower total dissolved solids with varied compositions of calcium, magnesium, bicarbonate, sulfate, sodium, and chloride.

To ensure the dispersibility of the binder composition across the country (and throughout the whole world), the binder composition may desirably be soluble in water containing up to about 100 ppm total dissolved solids and a CaCO₃ equivalent hardness up to about 55 ppm. More desirably, the binder composition may be soluble in water containing up to about 300 ppm of total dissolved solids and a CaCO₃ equivalent hardness up to about 150 ppm. Even more desirably, the binder composition may be soluble in water containing up to about 500 ppm total dissolved solids and a CaCO₃ equivalent hardness up to about 250 ppm.

### Triggerable Polymer

As previously discussed, the binder composition may comprise the triggerable polymer, and optionally an anti-blocking agent and/or a cobinder. A variety of triggerable polymers may be used. One type of triggerable polymer is a dilution triggerable polymer. Examples of dilution triggerable polymers include ion-sensitive polymers, which may be employed in combination with a wetting composition in which the insolubilizing agent is a salt. Other dilution triggerable polymers may also be employed, wherein these dilution triggerable polymers are used in combination with wetting agents using a variety of insolubilizing agents, such as organic or polymeric compounds.

Although the triggerable polymer may be selected from a variety of polymers, including temperature sensitive polymers and pH-sensitive polymers, the triggerable polymer may preferably be the dilution triggerable polymer, comprising the ion-sensitive polymer. If the ion-sensitive polymer is derived from one or more monomers, where at least one contains an anionic functionality, the ion-sensitive polymer is referred to as an anionic ion-sensitive polymer. If the ion-sensitive polymer is derived from one or more monomers, where at least one contains a cationic functionality, the ion-sensitive polymer is referred to as a cationic ion-sensitive polymer. An exemplary anionic ion-sensitive polymer is described in U.S. Pat. No. 6,423,804, which is incorporated herein in its entirety by reference.

Examples of cationic ion-sensitive polymers are disclosed in the following U.S. Patent Application Publication Nos.: 2003/0026963 A1; 2003/0027270 A1; 2003/0032352 A1; 2004/0030080 A1; 2003/0055146 A1; 2003/0022568 A1; 2003/0045645 A1; 2004/0058600 A1; 2004/0058073 A1; 2004/0063888 A1; 2004/0055704 A1; 2004/0058606 A1; and 2004/0062791 A1, and U.S. Pat. Nos. 7,070,854; 7,141,519; and 7,157,389, all of which are incorporated herein by reference in their entirety, except that in the event of any inconsistent disclosure or definition from the present application, the disclosure or definition herein shall be deemed to prevail.

Desirably, the ion-sensitive polymer may be insoluble in the wetting composition, wherein the wetting composition comprises at least about 0.3 weight percent of an insolubilizing agent which may be comprised of one or more inorganic and/or organic salts containing monovalent and/or divalent ions. More desirably, the ion-sensitive polymer may be insoluble in the wetting composition, wherein the wetting composition comprises from about 0.1% to about 10% by weight of an insolubilizing agent which may be comprised of one or more inorganic and/or organic salts containing monovalent and/or divalent ions. Even more desirably, the ion-sensitive polymer may be insoluble in the wetting composition, wherein the wetting composition comprises from about 0.5% to about 5% by weight of an insolubilizing agent which comprises one or more inorganic and/or organic salts containing monovalent and/or divalent ions. Especially desirably, the ion-sensitive polymer may be insoluble in the wetting composition, wherein the wetting composition comprises from about 1.0% to about 4.0% by weight of an insolubilizing agent which comprises one or more inorganic and/or organic salts containing monovalent and/or divalent ions. Suitable monovalent ions include, but are not limited to, Na⁺ ions, K⁺ ions, Li⁺ ions, NH₄⁺ ions, low molecular weight quaternary ammonium compounds (e.g., those having fewer than 5 carbons on any side group), and a combination thereof. Suitable divalent ions include, but are not limited to, Zn²⁺, Ca²⁺ and Mg²⁺. These monovalent and divalent ions may be derived from organic and inorganic salts including, but not limited to, NaCl, NaBr, KCl, NH₄Cl, Na₂SO₄, ZnCl₂, CaCl₂, MgCl₂, MgSO₄, and combinations thereof. Typically, alkali metal halides are the most desirable monovalent or divalent ions because of cost, purity, low toxicity, and availability. A particularly desirable salt is NaCl.

In a preferred embodiment, the ion-sensitive polymer may desirably provide the nonwoven web with sufficient in-use strength (typically >300 g/in.) in combination with the wetting composition containing sodium chloride. These nonwoven webs may be dispersible in tap water, desirably losing most of their wet strength (<100 g/in.) in 24 hours, or less.

In another preferred embodiment, the ion-sensitive polymer may comprise the cationic sensitive polymer, wherein the cationic sensitive polymer is a cationic polyacrylate that is the polymerization product of 96 mol % methyl acrylate and 4 mol % [2-(acryloyloxy)ethyl]trimethyl ammonium chloride.

### Cobinder Polymers

As previously discussed, the binder composition may comprise the triggerable polymer, and optionally an anti-blocking agent and/or a cobinder. When the binder composition comprises the triggerable polymer and the cobinder, the triggerable polymer and the cobinder may preferably be compatible with each other in aqueous solutions to: 1) allow for facile application of the binder composition to the fibrous substrate in a continuous process and 2) prevent interference with the dispersibility of the binder composition. Therefore, if the triggerable polymer is the anionic ion-sensitive polymer, cobinders which are anionic, nonionic, or very weakly cationic may be preferred. If the triggerable polymer is the cationic ion-sensitive polymer, cobinders which are cationic, nonionic, or very weakly anionic may be preferred. Additionally, the cobinder desirably does not provide substantial cohesion to the nonwoven material by way of covalent bonds, such that it interferes with the dispersibility of the nonwoven web.

The presence of the cobinder may provide a number of desirable qualities. For example, the cobinder may serve to reduce the shear viscosity of the triggerable polymer, such that the binder composition has improved sprayability over the triggerable binder alone. By use of the term “sprayable” it is meant that these polymers may be applied to the fibrous material or substrate by spraying, allowing the uniform distribution of these polymers across the surface of the substrate and penetration of these polymers into the substrate. The cobinder may also reduce the stiffness of the nonwoven web compared to the stiffness of a nonwoven web to which only the triggerable polymer has been applied. Reduced stiffness may be achieved if the cobinder has a glass transition temperature, T_{g}, that is lower than the T_{g} of the triggerable polymer. In addition, the cobinder may be less expensive than the triggerable polymer and by reducing the amount of triggerable polymer needed, may serve to reduce the cost of the binder composition. Thus, it may be desirable to use the highest amount of cobinder possible in the binder composition such that it does not jeopardize the dispersibility and in-use strength properties of the wet wipe. In a preferred embodiment, the cobinder replaces a portion of the triggerable polymer in the binder composition and permits a given strength level to be achieved, relative to a wet wipe having approximately the same tensile strength but containing only the triggerable polymer in the binder composition, to provide at least one of the following attributes: lower stiffness; better tactile properties (e.g. lubricity or smoothness); or reduced cost.

In one embodiment, the cobinder present in the binder composition, relative to the mass of the binder composition, may be about 10% or less, more desirably about 15% or less, more desirably 20% or less, more desirably 30% or less, or more desirably about 45% or less. Exemplary ranges of cobinder relative to the solid mass of the binder composition may include from about 1% to about 45%, from about 25% to about 35%, from about 1% to about 20% and from about 5% to about 25%.

The cobinder may be selected from a wide variety of polymers, as are known in the art. For example, the cobinder may be selected from the group consisting of poly(ethylene-vinyl acetate), poly(styrene-butadiene), poly(styrene-acrylic), a vinyl acrylic terpolymer, a polyester latex, an acrylic emulsion latex, poly vinyl chloride, ethylene-vinyl chloride copolymer, a carboxylated vinyl acetate latex, and the like. A variety of additional exemplary cobinder polymers are discussed in U.S. Pat. No. 6,653,406 and U.S. Patent Application Publication 2003/00326963, which are both incorporated herein by reference in their entirety.

### Anti-Blocking Agents and Anti-Blocking Coating Polymers

As noted above, the binder composition may optionally comprise an anti-blocking agent or anti-blocking coating. The anti-blocking agent and the anti-blocking coating are defined as polymeric materials that reduce or prevent the tendency of two adjacent layers of a material to stick together, particularly when under pressure or exposed to high ambient temperatures. In the case of wet wipes, the anti-blocking agent and the anti-blocking coating may desirably prevent the tendency of two adjacent sheets of wet wipe to adhere to one another, thereby reducing the sheet-to-sheet adhesion. Although the anti-blocking agent and the anti-blocking coating may be selected from similar polymeric materials, the anti-blocking agent and the anti-blocking coating may be distinguished based on how and when they are applied during formation of the wet wipe. The anti-blocking agent may preferably be applied to the fibrous substrate as a component of the binder composition, while the anti-blocking coating may preferably be applied to the surface of the nonwoven material, whether the nonwoven material is a nonwoven web or a nonwoven fabric.

Examples of suitable anti-blocking agents or anti-blocking coatings are described in, for example, Published U.S. Patent App. No. 2007/0141936.

### Wetting Composition

The wetting composition for use in combination with the nonwoven materials may desirably comprise an aqueous composition containing the insolubilizing agent that maintains the coherency of the binder composition and thus the in-use strength of the wet-wipe until the insolubilizing agent is diluted with tap water. Thus the wetting composition may contribute to the triggerable property of the triggerable polymer and concomitantly the binder composition.

Additionally, the preservative system of the present disclosure may be incorporated into the wetting composition. As noted above, the preservative system has efficacy against a broad spectrum of microorganisms. As such the preservative-containing wetting composition will help keep microbiological and fungal growth in the wet wipe at an acceptable level.

The insolubilizing agent in the wetting composition can be a salt, such as those previously disclosed for use with the ion-sensitive polymer, a blend of salts having both monovalent and multivalent ions, or any other compound, which provides in-use and storage strength to the binder composition and may be diluted in water to permit dispersion of the wet wipe as the binder composition transitions to a weaker state. The wetting composition may desirably contain more than about 0.1 weight percent of an insolubilizing agent based on the total weight of the wetting composition. The wetting composition may desirably contain from about 0.3 weight percent to about 10 weight percent of an insolubilizing agent based on the total weight of the wetting composition. More desirably, the wetting composition may contain from about 0.5 weight percent to about 5 weight percent of an insolubilizing agent based on the total weight of the wetting composition. More desirably, the wetting composition may contain from about 1 weight percent to about 4 weight percent of an insolubilizing agent based on the total weight of the wetting composition. Even more desirably, the wetting composition may contain from about 1 weight percent to about 2 weight percent of an insolubilizing agent based on the total weight of the wetting composition.

The wetting composition may desirably be compatible with the triggerable polymer, the cobinder polymer, the anti-blocking agent and any other components of the binder composition. In addition, the wetting composition desirably contributes to the ability of the wet wipes to maintain coherency during use, storage and/or dispensing, while still providing dispersibility in tap water.

The wetting composition may include a variety of additives or components, including those disclosed in U.S. Patent Publication No. 2002/0155281, which is incorporated herein in its entirety. Possible additives may include, but are not limited to skin-care additives, odor control additives, wetting agents and/or cleaning agents; water, emollients, surfactants, fragrances, preservatives, chelating agents, pH buffers, or combinations thereof as are well known to those skilled in the art. Further, the wetting agent may also contain lotions, medicaments, and/or other antimicrobials.

The wet wipes, as disclosed herein, do not require organic solvents to maintain in-use strength, and the wetting composition may be substantially free of organic solvents. Organic solvents may produce a greasy after-feel and cause irritation in higher amounts. However, small amount of organic solvents may be included in the wetting composition for different purposes other than maintaining in-use wet strength. In one embodiment, small amounts of organic solvents (less than about 1%) may be utilized as fragrance or preservative solubilizers to improve process and shelf stability of the wetting composition. The wetting composition may desirably contain less than about 5 weight percent of organic solvents, such as propylene glycol and other glycols, polyhydroxy alcohols, and the like, based on the total weight of the wetting composition. More desirably, the wetting composition may contain less than about 3 weight percent of organic solvents. Even more desirably, the wetting composition may contain less than about 1 weight percent of organic solvents.

Relative to the weight of the dry substrate, the wet wipe may desirably contain from about 10 percent to about 600 percent of the wetting composition by weight, more desirably from about 50 percent to about 500 percent of the wetting composition by weight, even more desirably from about 100 percent to about 400 percent of the wetting composition by weight, and especially more desirably from about 200 to 300 percent of the wetting composition.

### Method of Making Wet Wipes

The binder composition may be applied to the fibrous material by any known process. Suitable processes for applying the binder composition include, but are not limited to printing, spraying, electrostatic spraying, the use of metered press rolls or impregnating. The amount of binder composition may be metered and distributed uniformly onto the fibrous material or may be non-uniformly distributed onto the fibrous material.

For ease of application, the binder composition may be applied to the fibrous material in combination with a solvent, as a solution or mixture. A variety of solvents may be used, including, for example, water, methanol, ethanol, acetone, or the like, with water being the preferred solvent. The amount of binder composition in the solvent may vary, depending on a variety of factors, including the identity and physical characteristics of the triggerable polymer, the cobinder, and/or the anti-blocking agent that are being used, as well as the identity and physical characteristics of the fibrous material to which the binder composition is being applied. Desirably, the mixture or solution of the binder composition may contain up to about 50 percent by weight of binder composition solids. More desirably, the binder solution or mixture may contain from about 10 to 30 percent by weight of binder composition solids. Even more desirably, the binder solution or mixture may contain about 12 to 25 percent by weight binder composition solids.

Once the binder composition is applied to the fibrous material, drying, if necessary, may be achieved by any conventional means. Once dry, the nonwoven material may exhibit improved tensile strength when compared to the tensile strength of the untreated wet-laid or dry-laid fibrous material, and yet should have the ability to rapidly “fall apart” or disintegrate when placed in tap water.

A number of techniques may be employed to manufacture the wet wipes. In one embodiment, these techniques may include the following steps:

1. Providing the fibrous material (e.g., an unbonded airlaid, a tissue web, a carded web, fluff pulp, etc.).

2. Applying the binder composition to the fibrous material, typically in the form of a liquid, suspension, or foam to provide the nonwoven web

3. The nonwoven web may be dried.

4. The nonwoven web may be coated with a antiblocking coating composition in the form of a liquid, suspension, or foam.

5. Applying a wetting composition to the nonwoven web to generate the wet wipe.

6. Placing the wet wipe in roll form or in a stack and packaging the product.

In one embodiment, Step 2 as discussed above, may be carried out such that the triggerable polymer and the anti-blocking agent of the binder composition are applied as a mixture to the fibrous material, referred to as mixture application.

In another embodiment, Step 2 as discussed above, the application of the binder composition may be achieved by applying the triggerable polymer and the optional anti-blocking agent via different spray booms that are arranged sequentially, such that the triggerable polymer is applied first and the anti-blocking agent is applied second. This application technique may be referred to as a tandem or sequential application. That is, the fibrous material may travel past a plurality of spray booms, wherein a first set of spray booms applies the triggerable polymer and the second set of spray booms applies the anti-blocking agent, or vice-versa. This application technique may produce a layering effect of the triggerable polymer and the anti-blocking agent, preferably concentrating the anti-blocking agent on the surface of the nonwoven web.

In one embodiment, the binder composition as applied in step 2 may comprise the triggerable polymer. In another embodiment, the binder composition as applied in step 2 may comprise the triggerable polymer and an anti-blocking agent. In a further embodiment, the binder composition as applied in step 2 may comprise the triggerable polymer and a cobinder. Following step 3, the anti-blocking coating may be applied. Application of the anti-blocking coating may be achieved using a variety of techniques, including gravure printing, flexographic printing, inkjet printing, spray application and foam application, for example.

Wipes may also be prepared by applying the binder composition to the fibrous material, followed by drying, application of the anti-blocking coating (if desired) and winding of the resulting nonwoven web into a roll. In this embodiment, the wetting composition may be added some time later. For example, large rolls of the dry nonwoven web may be prepared as an intermediate material. This procedure may be advantageous as part of the manufacturing process. It may be desirable that blocking of the dry rolls or stacks of nonwoven web does not occur during storage, as such an occurrence would negatively impact unwinding of the rolls and subsequent converting of the dry basesheet into a wet wipe. Dry blocking can occur when the T_{g} of the binder composition in a nonwoven material is below or close to the storage temperature of the dry rolls of nonwoven materials.

The finished wet wipes may be individually packaged, desirably in a folded condition, in a moisture proof envelope or packaged in containers holding any desired number of sheets in a water-tight package with a wetting composition applied to the wipe. Some example processes which can be used to manufacture folded wet wipes are described in U.S. Pat. Nos. 5,540,332 and 6,905,748, which are incorporated by reference herein. The finished wipes may also be packaged as a roll of separable sheets in a moisture-proof container holding any desired number of sheets on the roll with a wetting composition applied to the wipes. The roll can be coreless and either hollow or solid. Coreless rolls, including rolls with a hollow center or without a solid center, can be produced with known coreless roll winders, including those of SRP Industry, Inc. (San Jose, Calif.); Shimizu Manufacturing (Japan), and the devices disclosed in U.S. Pat. No. 4,667,890. The U.S. Pat. No. 6,651,924 also provides examples of a process for producing coreless rolls of wet wipes.

### Wet Wipe Properties

The wet wipes, as disclosed herein, desirably may be made to have sufficient in-use wet tensile strength, wet thickness, opacity, and dispersibility. They may also be made to be usable without breaking or tearing, to be consumer acceptable, and provide problem-free disposal once disposed in a household sanitation system. Methods for measuring the wet wipe properties disclosed below are described in U.S. Published Patent App. No. 2007/0141936.

The wet wipe as disclosed herein desirably may have an in-use wet strength ranging from at least about 100 g/in to about 1000 g/in. More desirably, the wet wipe may have an in-use wet strength ranging from at least about 200 g/in to about 800 g/in. Even more desirably, the wet wipe may have an in-use wet strength ranging from at least about 300 g/in to about 600 g/in. Most desirably, the wet wipe may have an in-use wet strength ranging from at least about 350 g/in to about 550 g/in.

The wet wipe may be configured to provide all desired physical properties by use of a single or multi-ply wet wipe product, in which two or more plies of nonwoven material are joined together by methods known in the art to form a multi-ply wipe.

The total basis weight of the nonwoven material, consisting of a single or multiple layers of nonwoven material in the final wet wipe product, may be in the range of at least about 25 gsm to about 120 gsm. More desirably, the basis weight of the nonwoven material may be between about 40 gsm and 90 gsm. Even more desirably, the basis weight of the nonwoven material may be between about 60 gsm and 80 gsm. Especially more desirably, the basis weight of the nonwoven material may be between about 70 and 75 gsm.

The wet opacity of the wet wipe, or the tendency of the wet wipe to prevent the transmission of light, may desirably be higher (i.e. less transmitted light) as it provides an indication that the wet wipe will be able to perform its desired function without breaking or tearing.

Desirably, the wet wipe, as disclosed herein, may have a wet opacity greater than about 20%. More desirably, the wet wipe may have a wet opacity greater than about 35%. Even more desirably, the wet wipe may have a wet opacity greater than about 45%.

Preferably, the sheet-to-sheet adhesion of the wet wipe in the final packaged product may be lower, in order to provide easier dispensing of the wet wipe. Accordingly, the wet wipes, as disclosed herein, may desirably have a sheet-to-sheet adhesion less than about 7 g/in. More desirably, the wet wipes may have a sheet-to-sheet adhesion less than about 5 g/in. Even more desirably, the wet wipes may have a sheet-to-sheet adhesion less than about 3 g/in.

The average thickness of the wet wipe may be in the range of at least about 0.25 mm to about 1.5 mm. More desirably, the average thickness of the wet wipe may be between 0.3 mm and 1.0 mm. Even more desirably, the average thickness of the wet wipe may be between 0.5 mm and 1.0 mm.

As mentioned previously, the wet wipes, as disclosed herein, may be sufficiently dispersible so that they lose enough strength to break apart in tap water under conditions typically experienced in household or municipal sanitation systems. Also mentioned previously, the tap water used for measuring dispersibility should encompass the concentration range of the majority of the components typically found in the tap water compositions that the wet wipe would see upon disposal. Previous methods for measuring dispersibility of the nonwoven materials whether dry or pre-moistened, have commonly relied on systems in which the material was exposed to shear while in water, such as measuring the time for a material to break up while being agitated by a mechanical mixer. Constant exposure to such relatively high, uncontrolled shear gradients offers an unrealistic and overly optimistic test for products designed to be flushed in a toilet, where the level of shear is extremely weak or brief. Shear rates may be negligible, for example once the material enters a septic tank. Thus, for a realistic appraisal of wet wipe dispersibility, the test methods should simulate the relatively low shear rates the products will experience once they have been flushed in the toilet.

A static soak test, for example, should illustrate the dispersibility of the wet wipe after it is fully wetted with water from the toilet and where it experiences negligible shear, such as in a septic tank. Desirably, the wet wipe may have less than about 100 g/in of tensile strength after 5 h when soaked in water with a total dissolved solids up to 500 ppm and a CaCO₃ equivalent hardness up to about 250 ppm. More desirably, the wet wipe may have less than about 100 g/in of tensile strength after 3 h when soaked in water with a total dissolved solids up to 500 ppm and a CaCO₃ equivalent hardness up to about 250 ppm. Even more desirably, the wet wipe may have less than about 100 g/in of tensile strength after 1 h when soaked in water with a total dissolved solids up to 500 ppm and a CaCO₃ equivalent hardness up to about 250 ppm.

After flushing in the toilet in a household or building, the wet wipe may enter into the sanitary sewer system through pipes referred to as sewer laterals. In sewer laterals, the motion of the water typifies a “gentle sloshing” or wave-like motion. A “slosh box” is a box or a container that rocks back and forth with water inside, thereby creating a wave front and subjecting the wet wipe to intermittent motion that is capable of mimicking the “gentle sloshing” motion that the wet wipe would experience in sewer laterals. While the slosh box may be more vigorous than the actual action in a sewer lateral, the method is more representative of the lateral movement the wet wipe would experience than the higher shear methods described above. Desirably, the wet wipe will break-up in the slosh box to pieces of size less than about 1 inch square in area. Dispersion of the wet wipe to pieces of about this size or smaller may be sufficient to allow the pieces to pass through the bar screens typically found in municipal sanitary sewer treatment facilities and not cause problems or blockages in households.

In one embodiment, the wet wipe may break up into pieces of less than about 1 inch square in a slosh box in less than about 500 minutes in water with a total dissolved solids up to 500 ppm and a CaCO₃ equivalent hardness up to about 250 ppm. In another embodiment, the wet wipe may desirably break up into pieces of less than about 1 inch square in area in a slosh box in less than about 300 minutes in water with a total dissolved solids up to 500 ppm and a CaCO₃ equivalent hardness up to about 250 ppm. In a further embodiment, the wet wipe may more desirably break up into pieces of less than about 1 inch square in area in a slosh box in less than about 100 minutes in water with a total dissolved solids up to 500 ppm and a CaCO₃ equivalent hardness up to about 250 ppm. In another embodiment, the wet wipe may even more desirably break up into pieces of less than about 1 inch square in area in a slosh box in less than about 60 minutes in water with a total dissolved solids up to 500 ppm and a CaCO₃ equivalent hardness up to about 250 ppm.

Desirably, the wet wipes, as disclosed herein, may possess an in-use wet tensile strength of at least about 150 g/in when wetted with 10% to 400% of the wetting composition by weight relative to the weight of the nonwoven material, and a tensile strength of less than about 100 g/in when soaked in water with a total dissolved solids up to 500 ppm and a CaCO₃ equivalent hardness up to about 250 ppm after about 24 hours or less, desirably after about one hour.

Most desirably, the wet wipes, as disclosed herein, may possess an in-use wet tensile strength greater than about 300 g/in when wetted with 10% to 400% of the wetting composition by weight relative to the nonwoven material, and a tensile strength of less than about 100 g/in when soaked in water with a total dissolved solids up to 500 ppm and a CaCO₃ equivalent hardness up to about 250 ppm after about 24 hours or less, desirably after about one hour.

In a further embodiment, the wet wipes, as disclosed herein, may possess an in-use wet tensile strength greater than about 300 g/in when wetted with 10% to 400% of the wetting composition by weight relative to the weight of the nonwoven material, and a slosh box break-up time of less than about 300 minutes in water with a total dissolved solids up to 500 ppm and a CaCO₃ equivalent hardness up to about 250 ppm.

The wet wipe preferably maintains its desired characteristics over the time periods involved in warehousing, transportation, retail display and storage by the consumer. In one embodiment, shelf life may range from two months to two years.

Having described the disclosure in detail, it will be apparent that modifications and variations are possible without departing from the scope of the disclosure defined in the appended claims.

### EXAMPLES

The following non-limiting examples are provided to further illustrate the present disclosure.

### Test Methods

### Preservative Efficacy Test

The following test was used to determine the efficacy of the preservative system of the present disclosure at controlling various bacteria, yeast, and molds.

Test articles were prepared by incorporating a composition as set forth in Examples 1-5 below (including methylisothiazolinone and sodium benzoate) onto individual sheets of a dispersible, non-woven, airlaid basesheet, prepared as described in U.S. Publ. App. No. 2006/0008621, herein incorporated by reference. The dispersible binder composition used to form the basesheet comprised a cationic ion-sensitive polymer, specifically a cationic polyacrylate that was the polymerization product of 96 mol % methyl acrylate and 4 mol % [2-(acryloyloxy)ethyl]trimethyl ammonium chloride. The compositions from Examples 1-5 were incorporated onto the basesheets at a level equivalent to about 260% of the basesheet weight. The basesheets having the aqueous solution incorporated thereon were subjected to a sequence of six heating cycles, each cycle having a duration of about 90 seconds. The basesheets were allowed to cool to room temperature between each heating cycle for about three hours. The basesheets having the composition incorporated thereon were then subjected to a standardized protocol for testing preservative efficacy.

Prior to inoculation, the initial level of viable microorganism present in each test article was determined. One gram of test article was placed in 99 milliliters of diluent (either 0.9% saline solution or 0.9% saline solution containing 0.05% polysorbate 80 as noted below) and processed in a stomacher for one minute at medium speed. Ten milliliters of this suspension was plated between 3 plates containing a suitable agar material known in the art to represent a 1:10 test article dilution and 1.0 milliliter was transferred to an additional plate for a 1:100 dilution. Two separate sets of plates were prepared as above. One set was prepared and incubated for 3-5 days at from about 30° C. to about 35° C. for bacteria recovery. The second set of plates was prepared for yeast/mold recovery and was incubated for 5-7 days at from about 20° C. to about 25° C. After incubation a plate count was taken and multiplied by the dilution factor to determine the number of viable microorganisms present (in Colony Forming Units per gram of test article or CFU/g test article).

Six individual basesheets were inoculated with 0.1 milliliters of a challenge suspension, slightly spread over a one-gram area. Final challenge concentrations (in CFU/g test article) should be 1.0×10⁶ to 9.9×10⁶ bacteria inoculums, 1.0×10⁵ to 9.9×10⁵ yeast inoculum, or 1.0×10⁵ to 9.9×10⁵ mold inoculums. Bacteria inoculum suspensions were prepared in 0.9% saline solution for Escherichia coli and Enterobacter cloacae (1:1 mixture), Pseudomonas aeruginosa and Burkholderia cepacia (1:1 mixture), Burkholderia cepacia, and Staphylococcus aureus. Yeast inoculum suspensions were prepared in 0.9% saline solution for Candida albicans and mold inoculum suspensions were prepared in a 0.9% saline solution containing 0.05% polysorbate 80 for Aspergillus niger and Talaromyces luteus (1:1 mixture), for Mold Pool Isolates comprising Aureobasidium pullulans, Trichoderma harzianum, Penicillium corylophilum, and Alternaria alternata (1:1:1:1 mixture), and for Penicillium citrinum. The viable number of microorganisms can be each suspension is determined by any suitable plate count procedure known in the art, and the initial concentration of microorganisms per gram test article (O-hour inoculum level) was calculated as described above. The 0-hour inoculum level was used as a baseline for calculating the reduction in number of organisms over time. Inoculated test articles were maintained at from about 20° C. to about 25° C. for the duration of the study.

Enumeration of the remaining viable bacterial and fungal population in the inoculated test articles was conducted at various time intervals ranging from 1 day to 28 days post-inoculation. At each time interval, the inoculated one gram area of a single basesheet was aseptically removed and transferred to a sterile stomacher bag containing 99 milliliters of diluent (0.9% saline or 0.9% containing 0.05% polysorbate 80 as noted above). The test article was processed in a stomacher for one minute at medium speed. Ten milliliters of this suspension was distributed between 3 plates containing a suitable agar medium known in the art to represent the 1:10 test article dilution and 1.0 milliliters was transferred to an additional plate containing a suitable agar medium known in the art as the 1:100 dilution. Following incubation of the test plates as noted above, the plates were observed and the CFU's counted. The number of organisms observed was multiplied by the dilution factor of the plate to give the number of viable organism per gram of test article at each time interval.

The formulation was deemed adequately preserved if: (a) there was at least a 99.9% reduction of vegetative bacteria within 7 days following each challenge, and no increase for the duration of the test period; and (b) there was at least a 90% reduction of yeasts and molds within 7 days following each challenge, and no increase for the duration of the test period. The preservative was effective in the product if there was not less than a 2.0 log reduction in bacteria from the initial calculated count at 14 days, and no increase from the 14 day count at 28 days and there was no increase in the initial calculated count of yeast and mold at 14 and 28 days. No increase is defined as not more than 0.5 log₁₀ units higher than the previous values measured.

### Abbreviations

The following abbreviations are used in the examples:
SA=Staphylococcus aureus
Eco=Escherichia coli
Ecl=Enterobacter cloacae
PA=Pseudomonas aeruginosa
BC=Burkholderia cepacia
CA=Candida albicans
AN=Aspergillus niger
TL=Talaromyces luteus
MPI=Mold Pool Isolates
PC=Penicillium citrinum

### Examples 1-5

In these examples, wet wipe compositions comprising methylisothiazolinone and sodium benzoate were produced. The following components were used to prepare the compositions.

### Example 1

| Component | Weight % |
|---|---|
| Water | 96.1868 |
| Sodium chloride | 2.0000 |
| Aloe barbadensis leaf juice | 0.0025 |
| Sodium benzoate | 0.3000 |
| Sodium lauryl glucose carboxylate | 0.6400 |
| and lauryl glucoside | |
| Propylene glycol | 0.5000 |
| Methylisothiazolinone | 0.0097 |
| Laureth-7 citrate | 0.2000 |
| Fragrance (cocoon) | 0.0600 |
| Tocopheryl acetate | 0.0010 |
| Malic acid | 0.1000 |

### Example 2

| Component | Weight % |
|---|---|
| Water | 95.9268 |
| Sodium chloride | 2.0000 |
| Aloe barbadensis leaf juice | 0.0025 |
| Sodium benzoate | 0.4500 |
| Sodium lauryl glucose carboxylate | 0.6400 |
| and lauryl glucoside | |
| Propylene glycol | 0.5000 |
| Methylisothiazolinone | 0.0097 |
| Polysorbate-20 | 0.3000 |
| Fragrance (cocoon) | 0.0600 |
| Tocopheryl acetate | 0.0010 |
| Malic acid | 0.1100 |

### Example 3

| Component | Weight % |
|---|---|
| Water | 95.8178 |
| Sodium chloride | 2.0000 |
| Aloe barbadensis leaf juice | 0.0025 |
| Methylisothiazolinone | 0.0087 |
| Sodium lauryl glucose carboxylate | 0.6400 |
| and lauryl glucoside | |
| Propylene glycol | 0.5000 |
| Sodium benzoate | 0.4500 |
| Polysorbate-20 | 0.3000 |
| Fragrance (cocoon) | 0.0600 |
| Tocopheryl acetate | 0.0010 |
| Malic acid (liquid) | 0.2200 |

### Example 4

| Component | Weight % |
|---|---|
| Water | 95.8385 |
| Sodium chloride | 2.0000 |
| Aloe barbadensis leaf juice | 0.0025 |
| Methylisothiazolinone | 0.0080 |
| Sodium lauryl glucose carboxylate | 0.6400 |
| and lauryl glucoside | |
| Propylene glycol | 0.5000 |
| Sodium benzoate | 0.4500 |
| Polysorbate-20 | 0.3000 |
| Fragrance (cocoon) | 0.0600 |
| Tocopheryl acetate | 0.0010 |
| Malic acid (liquid) | 0.2000 |

### Example 5

| Component | Weight % |
|---|---|
| Water | 95.8395 |
| Sodium chloride | 2.0000 |
| Aloe barbadensis leaf juice | 0.0025 |
| Methylisothiazolinone | 0.0070 |
| Sodium lauryl glucose carboxylate | 0.6400 |
| and lauryl glucoside | |
| Propylene glycol | 0.5000 |
| Sodium benzoate | 0.4500 |
| Polysorbate-20 | 0.3000 |
| Fragrance (cocoon) | 0.0600 |
| Tocopheryl acetate | 0.0010 |
| Malic acid (liquid) | 0.2000 |

The compositions were prepared by combining the water, sodium chloride, and aloe barbadensis leaf juice together in a suitable sized glass beaker. The contents were stirred for 5 minutes. The methylisothiazolinone, sodium lauryl glucose carboxylate and lauryl glucoside surfactant, and propylene glycol were subsequently added, and the resulting mixture was stirred for 15 minutes. The sodium benzoate was added and the resulting mixture was stirred for 15 minutes, until uniform and free of crystals. The fragrance, tocopheryl acetate, and polysorbate 20 were separately premixed together. The two mixes were combined, and stirred for 10 minutes. The pH of the final composition was adjusted to the target pH of about 4.5±0.3 using the malic acid.

### Example 6

The effectiveness of compositions comprising methylisothiazolinone and sodium benzoate against various microorganisms was evaluated.

The formulation prepared in Example 1 was incorporated onto individual sheets prepared as described above in the Test Methods section, and was successfully tested for preservative efficacy using the methods noted above. The initial (pre-inoculation) amounts of bacteria, yeast, and mold present on the test articles was less than 10 CFU/g. Enumeration of the remaining viable bacterial and fungal population in the inoculated test articles was conducted at day 1, day 2, day 3, day 7, and day 15. Results of the preservative efficacy test confirm that the composition meets CTFA guidelines for preservative efficacy, described above. The results are shown in Table 1.

TABLE 1

| | Inoculum | | | | | |
|---|---|---|---|---|---|---|
| | 0 Hour | | | | | |
| | Placebo | 1 Day | 2 Day | 3 Day | 7 Day | 15 Day |
| Test | CFU/g | CFU/g | CFU/g | CFU/g | CFU/g | CFU/g |
| SA | 6.5 × 10⁶ | 8.2 × 10⁴ | <10 | <10 | <10 | <10 |
| Eco/Ecl | 8.1 × 10⁶ | 1.6 × 10⁴ | <10 | <10 | <10 | <10 |
| PA/BC | 8.2 × 10⁶ | 1.4 × 10⁵ | <10 | <10 | <10 | <10 |
| BC Isolate #8914 | 9.2 × 10⁶ | 1.7 × 10² | <10 | <10 | <10 | <10 |
| BC Isolate #8915 | 9.8 × 10⁶ | 3.9 × 10⁵ | <10 | <10 | <10 | <10 |
| CA | 5.9 × 10⁵ | 2.2 × 10⁵ | 3.1 × 10⁴ | <10 | <10 | <10 |
| AN/TL | 4.5 × 10⁵ | 1.5 × 10⁴ | 9.0 × 10⁴ | 4.5 × 10³ | 7.0 × 10¹ | <10 |
| MPI | 8.1 × 10⁵ | 1.0 × 10¹ | 6.0 × 10¹ | 9.0 × 10¹ | <10 | <10 |
| PC | 5.5 × 10⁵ | 1.0 × 10¹ | <10 | <10 | <10 | <10 |

To further test the efficacy of the composition against microorganisms, the preservative efficacy test was repeated on the same treated basesheets used for the initial challenge. In particular, the basesheets were reinoculated with the microorganisms, as described above, but further amounts of the preservative-containing composition were not applied to the basesheets prior to the reinoculation. Enumeration of the remaining viable bacterial and fungal population in the re-inoculated test articles was conducted at day 1, day 2, day 7, day 14, and day 28 post re-inoculation. The results of the rechallenge are set forth in Table 2.

TABLE 2

| | Inoculum | | | | | |
|---|---|---|---|---|---|---|
| | 0 Hour Placebo | 1 Day | 2 Day | 7 Day | 14 Day | 28 Day |
| Test | CFU/g | CFU/g | CFU/g | CFU/g | CFU/g | CFU/g |
| SA | 8.3 × 10⁶ | 3.6 × 10⁴ | <10 | <10 | <10 | <10 |
| Eco/Ecl | 9.4 × 10⁶ | 2.0 × 10⁴ | 2.0 × 10⁵ | <10 | <10 | <10 |
| PA/BC | 7.2 × 10⁶ | 3.3 × 10⁵ | 1.5 × 10³ | <10 | <10 | <10 |
| BC Isolate #8914 | 7.4 × 10⁶ | 2.6 × 10² | <10 | <10 | <10 | <10 |
| BC Isolate #8915 | 9.8 × 10⁶ | 5.6 × 10⁵ | 2.1 × 10² | <10 | <10 | <10 |
| CA | 5.5 × 10⁵ | 3.7 × 10⁵ | 1.8 × 10⁴ | <10 | <10 | <10 |
| AN/TL | 3.7 × 10⁵ | 6.5 × 10⁴ | 2.4 × 10⁵ | 1.3 × 10² | <10 | <10 |
| MPI | 2.9 × 10⁵ | 1.2 × 10¹ | 2.0 × 10² | 1.3 × 10² | <10 | <10 |
| PC | 9.1 × 10⁵ | * | <10 | 1.0 × 10¹ | <10 | <10 |

* count unavailable-plates missing

As can be seen from these results, the preservative-containing composition maintained effectiveness against S. aureus, E. coli, E. cloacae, P. aeruginosa, C. albicans, A. niger, T. luteus, the Mold Pool Isolates, B. cepacia, and P. citrinum even upon rechallenge, indicating that the composition exceeds CTFA preservative efficacy guidelines for these microorganisms.

### Example 7

The effectiveness of compositions comprising methylisothiazolinone and sodium benzoate against various microorganisms was evaluated.

The composition prepared in Example 2 was incorporated onto individual sheets prepared as described above in the Test Methods section, and was successfully tested for preservative efficacy using the methods noted above. The initial (pre-inoculation) amounts of bacteria, yeast, and mold present on the test articles was less than 10 CFU/g. Enumeration of the remaining viable bacterial and fungal population in the inoculated test articles was conducted at day 1, day 2, day 3, day 7, and day 15. Results of the preservative efficacy test confirm that the composition meets CTFA guidelines for preservative efficacy, described above. The results are shown in Table 3.

TABLE 3

| | Inoculum | | | | | |
|---|---|---|---|---|---|---|
| | 0 Hour | | | | | |
| | Placebo | 1 Day | 2 Day | 3 Day | 7 Day | 15 Day |
| Test | CFU/g | CFU/g | CFU/g | CFU/g | CFU/g | CFU/g |
| SA | 6.5 × 10⁶ | 1.2 × 10⁵ | 3.0 × 10¹ | <10 | <10 | <10 |
| Eco/Ecl | 8.1 × 10⁶ | 1.1 × 10³ | <10 | <10 | <10 | <10 |
| PA/BC | 8.2 × 10⁶ | 7.6 × 10⁴ | <10 | <10 | <10 | <10 |
| BC Isolate #8914 | 9.2 × 10⁶ | <10 | <10 | <10 | <10 | <10 |
| BC Isolate #8915 | 9.8 × 10⁶ | 9.6 × 10⁴ | <10 | <10 | <10 | <10 |
| CA | 5.9 × 10⁵ | 2.3 × 10⁵ | 1.2 × 10⁴ | <10 | <10 | <10 |
| AN/TL | 4.5 × 10⁵ | 2.0 × 10³ | 1.3 × 10⁴ | 1.2 × 10³ | 7.0 × 10¹ | <10 |
| MPI | 8.1 × 10⁵ | 6.4 × 10² | 1.0 × 10¹ | 1.0 × 10¹ | <10 | <10 |
| PC | 5.5 × 10⁵ | <10 | <10 | <10 | <10 | <10 |

To further test the efficacy of the composition against microorganisms, the preservative efficacy test was repeated on the same treated basesheets used for the initial challenge. In particular, the basesheets were reinoculated with the microorganisms, as described above, but further amounts of the preservative-containing composition were not applied to the basesheets prior to the reinoculation. Enumeration of the remaining viable bacterial and fungal population in the re-inoculated test articles was conducted at day 1, day 2, day 7, day 14, and day 28 post re-inoculation. The results of the rechallenge are set forth in Table 4.

TABLE 4

| | Inoculum | | | | | |
|---|---|---|---|---|---|---|
| | 0 Hour Placebo | 1 Day | 2 Day | 7 Day | 14 Day | 28 Day |
| Test | CFU/g | CFU/g | CFU/g | CFU/g | CFU/g | CFU/g |
| SA | 8.3 × 10⁶ | 1.9 × 10⁵ | 1.8 × 10³ | <10 | <10 | <10 |
| Eco/Ecl | 9.4 × 10⁶ | 7.3 × 10⁴ | <10 | <10 | <10 | <10 |
| PA/BC | 7.2 × 10⁶ | 1.1 × 10⁵ | <10 | <10 | <10 | <10 |
| BC Isolate #8914 | 7.4 × 10⁶ | 4.1 × 10² | <10 | <10 | <10 | <10 |
| BC Isolate #8915 | 9.8 × 10⁶ | 5.1 × 10⁵ | 5.0 × 10¹ | <10 | <10 | <10 |
| CA | 5.5 × 10⁵ | 5.8 × 10⁵ | 9.2 × 10³ | <10 | <10 | <10 |
| AN/TL | 3.7 × 10⁵ | 5.1 × 10⁴ | 2.9 × 10³ | 9.0 × 10¹ | <10 | <10 |
| MPI | 2.9 × 10⁵ | 1.4 × 10² | 1.4 × 10² | 1.0 × 10¹ | <10 | <10 |
| PC | 9.1 × 10⁵ | 9.9 × 10⁴ | 4.0 × 10¹ | <10 | <10 | <10 |

As can be seen from these results, the preservative-containing composition maintained effectiveness against S. aureus, E. coli, E. cloacae, P. aeruginosa, C. albicans, A. niger, T. luteus, the Mold Pool Isolates, B. cepacia, and P. citrinum even upon rechallenge, indicating that the composition exceeds CTFA preservative efficacy guidelines for these microorganisms.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products without departing from the scope of the disclosure, it is intended that all matter contained in the above description shall be interpreted as illustrative and not in a limiting sense.

What is claimed is:

1. A composition comprising methylisothiazolinone and a benzoic acid salt or derivative thereof at a weight ratio of methylisothiazolinone to benzoic acid salt or derivative thereof of from about 1:30 to about 1:100, wherein the composition has a pH of about 6.0 or less.

2. The composition of claim 1 wherein the weight ratio of methylisothiazolinone to benzoic acid salt or derivative thereof is from about 1:50 to about 1:70.

3. The composition of claim 1 wherein the pH of the composition is from about 4.2 to about 5.8.

4. The composition of claim 1 wherein the composition comprises from about 0.005% (w/w) to about 0.01% (w/w) of methylisothiazolinone.

5. The composition of claim 4 wherein the composition comprises from about 0.0070% (w/w) to about 0.0095% (w/w) of methylisothiazolinone.

6. The composition of claim 1 wherein the composition comprises from about 0.30% (w/w) to about 0.50% (w/w) of benzoic acid salt or derivative thereof.

7. The composition of claim 1 wherein the benzoic acid salt or derivative thereof is selected from the group consisting of sodium benzoate, potassium benzoate, and combinations thereof.

8. The composition of claim 7 comprising from about 0.005% (w/w) to about 0.01% (w/w) of methylisothiazolinone and from about 0.30% (w/w) to about 0.50% (w/w) of sodium benzoate.

9. The composition of claim 1 further comprising from about 0.1% (w/w) to about 99% (w/w) of water.

10. The composition of claim 1 further comprising a component selected from the group consisting of water, emollients, sterols or sterol derivatives, natural and synthetic fats or oils, viscosity enhancers, rheology enhancers, polyols, surfactants, alcohols, esters, silicones, clays, starch, cellulose, and combinations thereof.

11. A wet wipe comprising a wipe substrate and a liquid composition, the liquid composition comprising methylisothiazolinone and a benzoic acid salt or derivative thereof at a weight ratio of methylisothiazolinone to benzoic acid salt of from about 1:30 to about 1:100, wherein the liquid composition has a pH of about 6.0 or less.

12. The wet wipe of claim 11 wherein the weight ratio of methylisothiazolinone to benzoic acid salt or derivative thereof in the liquid composition is from about 1:50 to about 1:70.

13. The wet wipe of claim 11 wherein the pH of the liquid composition is from about 4.2 to about 5.8.

14. The wet wipe of claim 11 wherein the liquid composition comprises from about 0.005% (by total weight of the composition) to about 0.01% (by total weight of the composition) of methylisothiazolinone.

15. The wet wipe of claim 14 wherein the liquid composition comprises from about 0.0070% (by total weight of the composition) to about 0.0095% (by total weight of the composition) of methylisothiazolinone.

16. The wet wipe of claim 11 wherein the liquid composition comprises from about 0.30% (by total weight of the composition) to about 0.50% (by total weight of the composition) of benzoic acid salt or derivative thereof.

17. The wet wipe of claim 11 wherein the benzoic acid salt is selected from the group consisting of sodium benzoate, potassium benzoate, and combinations thereof.

18. The wet wipe of claim 17 wherein the liquid formulation comprises from about 0.005% (by total weight of the composition) to about 0.01% (by total weight of the composition) of methylisothiazolinone and from about 0.30% (by total weight of the composition) to about 0.50% (by total weight of the composition) of sodium benzoate.

19. The wet wipe of claim 11 wherein the liquid composition further comprises from about 0.1% (by total weight of the composition) to about 99% (by total weight of the composition) of water.

20. The wet wipe of claim 11 wherein the liquid composition further comprises an additional component selected from the group consisting of water, emollients, surfactants, fragrances, preservatives, chelating agents, pH buffers, or combinations thereof.
